(19) United States
(12) Patent Application Publication
(10) Pub. No.: US 2026/0142036 A1
(43) Pub. Date: May 21, 2026

(54) Machine Learning-Based Risk Stratification and Management of Non-Alcoholic Fatty Liver Disease

(71) Applicant: Mayo Foundation for Medical Education and Research, Rochester, MN (US)

(72) Inventors: Alina M. Allen, Rochester, MN (US); Nan Zhang, Sewickley, PA (US); Shivaram Poigai Arunachalam, Rochester, MN (US)

(21) Appl. No.: 19/121,032

(22) PCT Filed: Nov. 3, 2023
(86) PCT No.: PCT/US2023/078689

§ 371 Date: Apr. 14, 2025

Related U.S. Application Data
(60) Provisional application No. 63/382,526, filed on Nov. 6, 2022.

(51) Int. Cl. G16H 50/30; G06N 3/0464; G06N 20/00; G16H 10/60

(52) U.S. Cl. CPC G16H 50/30; G06N 3/0464; G06N 20/00; G16H 10/60

(57) ABSTRACT

Screening and risk-stratification of patients at risk for developing liver disease, such as non-alcoholic fatty liver disease (“NAFLD”), among others, is achieved by applying an optimized set of patient health data features to a suitably trained machine learning algorithm or model. The machine learning model outputs NAFLD risk score data that quantify or otherwise indicate a risk of the patient developing NAFLD based on features present in their patient health data. The NAFLD risk score data can be further analyzed to risk stratify the patient and assist with determining next steps in the healthcare workflow for the patient.

### BACKGROUND

Nonalcoholic fatty liver disease (“NAFLD”) has become the most common cause of chronic liver disease in the industrialized countries and a major public health problem due to the unrelenting challenge of obesity. Based on extensive data, including a recent meta-analysis, the estimated prevalence of NAFLD in the United States is approximately 24%, thus affecting 80 million adults. NAFLD leads to higher mortality and increased risk of liver-related complications resulting in the need for liver transplantation as the only cure. As the prevalence of NAFLD is estimated to rise to 30%, the healthcare burden and resource utilization associated with the care of these patients will become increasingly high.

Currently, the identification of NAFLD patients relies heavily on primary care/family medicine providers. However, due to lack of easily applicable screening methods, limited awareness and time allotted to cover this topic beyond the presenting complaints, most patients remain unidentified. Moreover, even when identified, there are no cost-effective methods to risk-stratify patients with significant liver disease (fibrosis), who need to be referred to hepatology from those with early disease, who can be managed in primary care. Consequently, due to the lack of symptoms and universal screening policies, many individuals remain undiagnosed until late, when they develop signs and symptoms of end-stage liver disease, and the disease is irreversible.

NAFLD outcomes would be improved with early diagnosis and timely management because the disease is reversible at early stages. Methods for large scale pre-screening and identification of individuals with NAFLD are urgently needed, to allow timely intervention, improve patient outcomes while also reducing healthcare costs. Additionally, risk-stratification and prediction of a progressive NAFLD phenotype are major unmet needs. The mere presence of fat in the liver is not sufficient to predict future development of liver disease. In fact, only 1-2% of individuals diagnosed with NAFLD will advance to cirrhosis and complications, while the remainder will have increased mortality due to non-liver related complications, mainly cardiovascular disease and cancers. Hence, once NAFLD is identified, a second step of risk stratification would distinguish those who are destined to progress to end-stage liver disease (and need close surveillance and aggressive intervention as a priority) from those who may not need intensive monitoring for cirrhosis and complications.

### SUMMARY OF THE DISCLOSURE

The present disclosure addresses the aforementioned drawbacks by providing a method for risk stratifying a patient for non-alcoholic fatty liver disease (“NAFLD”) using machine learning. The method includes accessing patient health data for a patient with a computer system, and accessing a machine learning model with the computer system. The machine learning model has been trained on training data in order to generate NAFLD risk scores based on features present in a patient's patient health data. The patient health data are applied to the machine learning model, generating an output as NAFLD risk score data that indicate a risk of the patient developing NAFLD based on features in their patient health data.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for generating NAFLD risk score data by inputting a patient's patient health data to a suitably trained machine learning model.

FIG. 2 is a feature importance plot indicating the relative importance of various patient health data features as they relate to risk stratification of NAFLD.

FIG. 3 is a flowchart setting forth the steps of an example method for training a machine learning model to generate NAFLD risk score data from input patient health data.

FIG. 4 is a block diagram of an example NAFLD risk scoring system in accordance with some embodiments described in the present disclosure.

FIG. 5 is a block diagram of example components that can implement the system of FIG. 4.

### DETAILED DESCRIPTION

Described here are systems and methods for screening and risk-stratifying patients at risk for developing liver disease, such as non-alcoholic fatty liver disease (“NAFLD”) among others, based on inputting an optimized set of features from patient health data into a suitably trained machine learning algorithm or model.

Machine learning provides a promising solution to process enormous amounts of data points that exceed the performance of human expertise in interpretation. Suitably trained machine learning models can offer a practical solution to large scale implementation of screening and risk-stratification strategies. Using patient health data that are routinely collected, the systems and methods described in the present disclosure enable providers in any non-hepatology area to identify patients with NAFLD, or other liver diseases, via an automated machine learning model that can be embedded in the EHR system. The machine learning model is trained to assess the patient's risk of NAFLD and to alert the clinician if that patient's risk is high. Using a cutoff of predicted risk, a clinical model of care can be embedded in the flow to assist decision making, such as by referring the patient for detailed evaluation of liver disease and identification of patients who are in need of aggressive intervention.

Electronic health record datasets include very large numbers of observations, which can deliver a rich predictive power, but require careful and complex computational considerations due to several aspects. One challenge with EHR and other patient health data is that the inputs are mixtures of quantitative, binary, and categorical variables, the latter often with many levels. Patient health data can be challenging to work with because there are also often complex interactions between variables and/or features, such as medications and labs or diagnoses. Furthermore, there are often many missing values and outliers, reflective of real-world data. For instance, a particular patient may have an associated set of patient health data that may not have all of the same data values or types as a training dataset acquired from a large cohort of patients (e.g., a patient whose data will be input to a trained model may be missing a particular lab value that may have been largely present in the training data set). Additionally or alternatively, only a small fraction of the large number of predictor variables are actually relevant to prediction; hence, traditional statistical methods such as linear or logistic regression do not afford the necessary computational scalability.

A variety of machine learning methods can be used for predictive learning from data mining, such as decision tree-based methods, neural networks, and so on. In diseases such as NAFLD, with highly non-linear and complex relationship between features and outcomes, decision tree-based methods such as random forests and gradient boosting machines (“GBM”) are advantageous for handling complex EHR features because of their ability to model interactions and automatically select relevant variables, as well as their robustness to outliers and missing data. On the other hand, the predictive power of these machine learning models may not be as high as that of neural networks, which have the disadvantage of not being able to handle missing data as readily as decision tree-based methods.

Thus, in some embodiments, a decision tree-based method, such as random forest or GBM, can be used. In still other embodiments, a neural network, such as a convolutional neural network, can be used. In yet other embodiments, a decision tree-based method, such as GBM, can be used in a first step to identify the features or variables of highest importance, which can then be included as features in a neural network model to achieve higher predictive performance. Additionally or alternatively, strategies to better adapt the patient health data to a neural network model can be used. As one example, missing values whose effect was found to be structural can be represented as explicit indicator variables.

Following implementation of the machine learning model, the individual's risk of NAFLD or other liver diseases or conditions (e.g., advanced fibrosis) can be automatically generated and available for interpretation to any providers, including those in non-hepatology areas, such as primary care/family medicine, endocrinology, and cardiology. Those with high risk for NAFLD fibrosis can be recommended to undergo further evaluation with elastography and/or can be referred to specialty care (e.g., gastroenterology and hepatology) for aggressive management to prevent further disease progression. The machine learning model can generate the risk-score at each time point of healthcare, therefore the model can be updated longitudinally. Those with NAFLD but low risk of fibrosis can benefit from intervention to promote weight loss, but without the need for specialized testing with elastography or referral to specialty care. Identification of early NAFLD and timely intervention to lose weight decreases the risk of complications associated with NAFLD, including cirrhosis, liver cancer, need for liver transplantation, cardiovascular disease, and extrahepatic cancers.

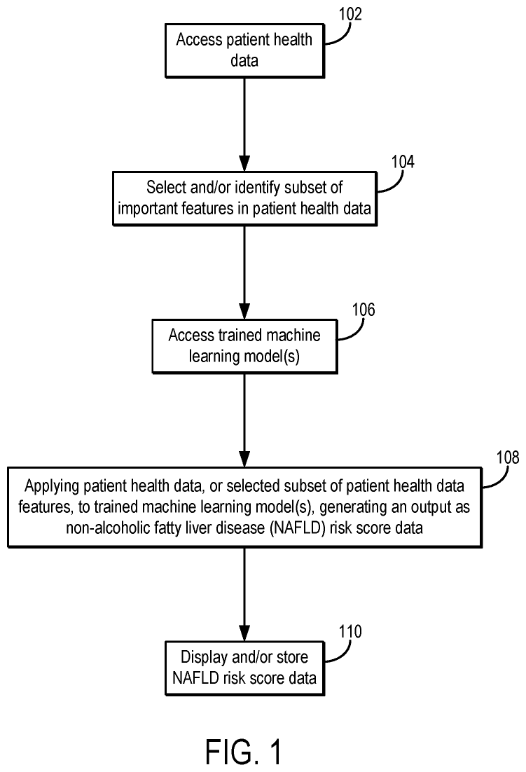

Referring now to FIG. 1, a flowchart is illustrated as setting forth the steps of an example method for generating NAFLD risk score data using a suitably trained neural network or other machine learning algorithm. As will be described, the neural network or other machine learning algorithm takes patient health data as input data and generates NAFLD risk score data as output data. As an example, the NAFLD risk score data can include a percent score or probability for being at risk for NAFLD, a numerical score, and/or a categorical indicator (e.g., “high” risk, “moderate” risk, “low” risk). Additionally or alternatively, the NAFLD risk score data can include a quantitative estimate of tissue and/or organ damage, such as how severe damage is, a stage of scar tissue, the presence of liver cirrhosis, and so on.

The method includes accessing patient health data with a computer system, as indicated at step 102. Accessing the patient health data may include retrieving such data from a memory or other suitable data storage device or medium.

The patient health data may include data stored in, retrieved from, extracted from, or otherwise derived from the patient's electronic medical record (“EMR”) and/or electronic health record (“EHR”). The patient health data can include unstructured text, questionnaire response data, clinical laboratory data, histopathology data, genetic sequencing, medical imaging, and other such clinical data types. Examples of clinical laboratory data and/or histopathology data can include genetic testing and laboratory information, such as performance scores, lab tests, pathology results, prognostic indicators, date of genetic testing, testing method used, and so on.

In some instances, the patient health data can include one or more types of omics data, such as genomics data, proteomics data, transcriptomics data, epigenomics data, metabolomics data, microbiomics data, and other multiomics data types. The patient health data can additionally or alternatively include patient geographic data, demographic data, and the like. In some instances, the patient health data can include information pertaining to diagnoses, responses to treatment regimens, genetic profiles, clinical and phenotypic characteristics, and/or other medical, geographic, demographic, clinical, molecular, or genetic features of the patient.

Features derived from structured, curated, and/or EMR or EHR data may include clinical features such as diagnoses; symptoms; therapies; outcomes: patient demographics, such as patient name, date of birth, gender, and/or ethnicity; diagnosis dates for cancer, illness, disease, or other physical or mental conditions; personal medical history; family medical history: clinical diagnoses, such as date of initial diagnosis, date of metastatic diagnosis, cancer staging, tumor characterization, and tissue of origin; and the like. Additionally, the patient health data may also include features such as treatments and outcomes, such as line of therapy, therapy groups, clinical trials, medications prescribed or taken, surgeries, radiotherapy, imaging, adverse effects, and associated outcomes.

Patient health data can include a set of clinical features associated with information derived from clinical records of a patient, which can include records from family members of the patient. These clinical features and data may be abstracted from unstructured clinical documents, EMR, EHR, or other sources of patient history. Such data may include patient symptoms, diagnosis, treatments, medications, therapies, responses to treatments, laboratory testing results, medical history, geographic locations of each, demographics, or other features of the patient which may be found in the patient's EMR and/or EHR.

In some instances, patient health data can include medical imaging data, which may include images of the patient obtained with one or more different medical imaging modalities, including magnetic resonance imaging (“MRI”), computed tomography (“CT”), x-ray imaging, positron emission tomography (“PET”), ultrasound, and so on. The medical imaging data may also include parameters or features computed or derived from such images. Medical imaging data may also include digital pathology images, such as H&E slides, IHC slides, and the like. The medical imaging data may also include data and/or information from pathology and radiology reports, which may be ordered by a physician during the course of diagnosis and treatment of various illnesses and diseases.

As a non-limiting example, epigenomics data may include data associated with information derived from DNA modifications that are not changes to the DNA sequence and regulate the gene expression. These modifications can be a result of environmental factors based on what the patient may breathe, eat, or drink. These features may include DNA methylation, histone modification, or other factors which deactivate a gene or cause alterations to gene function without altering the sequence of nucleotides in the gene.

Microbiomics data may include, for example, data derived from the viruses and bacteria of a patient. These features may include viral infections which may affect treatment and diagnosis of certain illnesses as well as the bacteria present in the patient's gastrointestinal tract which may affect the efficacy of medicines ingested by the patient.

Proteomics data may include data associated with information derived from the proteins produced in the patient. These features may include protein composition, structure, and activity; when and where proteins are expressed; rates of protein production, degradation, and steady-state abundance; how proteins are modified, for example, post-translational modifications such as phosphorylation; the movement of proteins between subcellular compartments; the involvement of proteins in metabolic pathways; how proteins interact with one another: or modifications to the protein after translation from the RNA such as phosphorylation, ubiquitination, methylation, acetylation, glycosylation, oxidation, or nitrosylation.

Genomics data may include genomic info that can be, or have been, correlated with the symptoms and medication effect, tolerance, and/or side effect information that may be received from a patient as responses to a questionnaire and stored as questionnaire response and/or phenotypic data. As anon-limiting example, genomics data can be extracted from blood or saliva samples collected from individuals who have also completed one or more questionnaires such that corresponding questionnaire response data is available for the individuals. A deep phenotypic characterization of these individuals can be assembled. As an example, in one large subset, prospectively determined patterns of treatment response after protocoled titrations in various different drugs from distinct classes of treatments have been assembled. For instance, an analysis of Verapamil, (an L-type calcium channel blocker) using whole exome sequencing (“WES”) can be completed following genotyping in a confirmatory cohort.

In some embodiments, the patient health data can include a collection of data and/or features including all of the data types disclosed above. Alternatively, the patient health data may include a selection of fewer data and/or features.

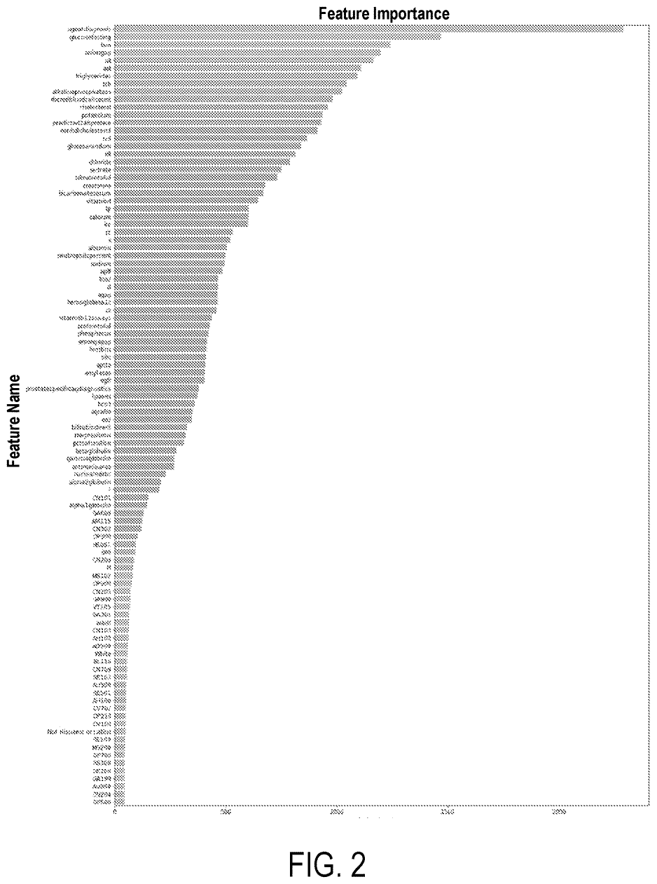

As indicated at step 104, in some embodiments a subset of features that have been identified as having higher importance or relevance to risk stratifying NAFLD can be selected from the acquired patient health data. As a non-limiting example, the features may include patient age at diagnosis, prostate specific diagnostics, gender (male or female), body mass index (“BMI”), waist circumference, and ethnicity (e.g., Caucasian, not Hispanic or Latino, etc.), blood test results, and whether the patient is currently prescribed and/or taking certain medications. Example of these features and their relative importance are illustrated in FIG. 2. In some embodiments, the subset of features can be selected using a machine learning algorithm, such as a decision tree-based method that ranks the importance of features in the patient health data across a large cohort of patients.

Blood test results may include glucose levels obtained while fasting, blood urea nitrogen (“BUN”) (i.e., an amount of urea nitrogen in the patient's blood), anion gap (i.e., a measure of the difference between negatively and positively charged electrolytes in the patient's blood) (“AGAP”), alanine transaminase (“ALT”), aspartate transferase (“AST”), triglycerides, thyroid-stimulating hormone (“TSH”), alkaline phosphatase (“ALP”), red blood cell count (“RBC”), cholesterol, potassium (“K”), predicted 24 hour protein, non-high-density lipoprotein (“HDL”) cholesterol, HDL, random glucose (i.e., glucose measured without fasting), low-density lipoprotein (“LDL”), chloride, erythrocyte sedimentation rate (“ESR”), bilirubin total, creatinine, bicarbonate serum, vitamin D, total protein (“TP”), calcium, international normalized ratio (“INR”), prothrombin time (“PT”), albumin, neutrophils percent, sodium, activated partial thromboplastin (“aPTT”), total carbon dioxide (“TCO2”). D-dimer (“d”), hemoglobin A1C, creatine kinase (“CK”), vitamin B12 assays, phosphorus, anion gap serum/plasma (“aniongapsp”), ferritins, total iron-binding capacity (“TIBC”), activated partial thromboplastin time (plasma) (“APTTP”), amylases, estimated glomerular filtration rate (“eGFR”), lipases, bicarbonate (“HCO3”), albumin/globulin (A/G) ratio, carbon dioxide (“CO2”), bilirubin direct, magnesiums, procalcitonin test (“PCT”), beta globulin, gamma globulin, antinuclear antibody (“ANA”), nucleated RBC, alpha 2 globulin, and alpha 1 globulin.

Medications can be referenced by national drug file reference terminology (“NDF-RT”) codes for various medications present in the patient's blood or otherwise prescribed to or being taken by the patient:

| NDF-RT Code | Medication Description |
|---|---|
| CN101 | Opioid analgesics |
| GA605 | Antiemetics |
| AM115 | Cephalosporin, 1st generation |
| CN302 | Benzodiazepine derivative sedatives/hypnotics |
| OP300 | Anti-inflammatories topical ophthalmic |
| HS051 | Glucocorticoids |
| CN203 | General anesthetics, other |
| MS102 | Nonsalicylate NSAIs, anti-rheumatic |
| OP900 | Ophthalmics, other |
| CN205 | Anesthetic adjuncts |
| GA900 | Gastric medications, other |
| VT105 | Thiamine |
| GA301 | Histamine antagonists |
| CN103 | Non-opioid analgesics |
| AH102 | Antihistamines, ethanolamine |
| AD900 | Antidotes/deterrents, other |
| BL110 | Anticoagulants |
| CN709 | Antipsychotics, other |
| RE103 | Bronchodilators, sympathomimetic, oral |
| AU300 | Parasympathomimetics (cholinergics) |
| RE501 | Antihistamine/decongestant |
| AH100 | Antihistamines, phenothiazine |
| CV702 | Loop diuretics |
| OP210 | Antibacterials, topical ophthalmic |
| CV100 | Beta blockers/related |
| RE109 | Antiasthma, other |
| MS200 | Skeletal muscle relaxants |
| OP700 | Anesthetics, topical ophthalmic |
| RS300 | Laxatives, rectal |
| DE200 | Anti-inflammatory, topical |
| GA199 | Antacids, other |
| AU350 | Parasympatholytics |
| CN204 | Local anesthetics, injection |
| OP500 | Eye washes/lubricants |

One or more trained machine learning models (e.g., a random forest model, a GBM model, a neural network) are then accessed with the computer system, as indicated at step 106. Accessing the trained machine learning model may include accessing model parameters (e.g., weights, biases, or both) that have been optimized or otherwise estimated by training the machine learning model on training data. In some instances, retrieving the machine learning model can also include retrieving, constructing, or otherwise accessing the particular model architecture to be implemented.

For instance, when the machine learning model is a neural network, data pertaining to the layers in the neural network architecture (e.g., number of layers, type of layers, ordering of layers, connections between layers, hyperparameters for layers) may be retrieved, selected, constructed, or otherwise accessed. An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value: an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the artificial neural network estimates a NAFLD risk score, the output layer may include a single node corresponding to a probability risk score value, a percent risk score value, a numerical risk score value, or a risk category label. In an example in which the artificial neural network quantifies an estimate of tissue and/or organ damage, the output layer may include one or more nodes, where each different node corresponds to a different quantitative estimate of severity. A first node may indicate severity (e.g., mild, moderate, advanced), a second node may indicate scar tissue stage, and so on.

The patient health data are then input to the one or more machine learning models, generating output as NAFLD risk score data, as indicated at step 108. As described above, in some embodiments only a subset of the patient health data pertaining to features identified as important or otherwise relevant for NAFLD risk stratification are input to the machine learning model(s). The NAFLD risk score data can provide physicians or other clinicians with a recommendation to consider additional monitoring for subjects whose patient health data indicate the likelihood of the subject developing or otherwise having NAFLD or other liver disease. For instance, the NAFLD risk score data can include a percent score or probability for being at risk for NAFLD, a numerical score, and/or a categorical indicator (e.g., “high” risk, “moderate” risk, “low” risk). As an example, the NAFLD risk score data can include a probability the patient health data include patterns, features, or characteristics indicative of detecting, differentiating, and/or determining the severity of NAFLD. Additionally or alternatively, the NAFLD risk score data can include a quantitative estimate of tissue and/or organ damage, such as how severe damage is, a stage of scar tissue, the presence of liver cirrhosis, and so on.

The NAFLD risk score data generated by inputting the patient health data to the trained machine learning model(s) can then be displayed to a user, stored for later use or further processing, or both, as indicated at step 110. As described above, in some embodiments the NAFLD risk score data can be analyzed by a computer system to generate an order set for follow up examination of the patient. For example, if the NAFLD risk score data indicate the patient is at high risk for NAFLD, an order set for further examination including elastography studies, or the like, can be generated and entered into the EHR system to order the further testing for the patient. Additionally or alternatively, the order set may also include less invasive orders or suggestions for the patient, including weight loss.

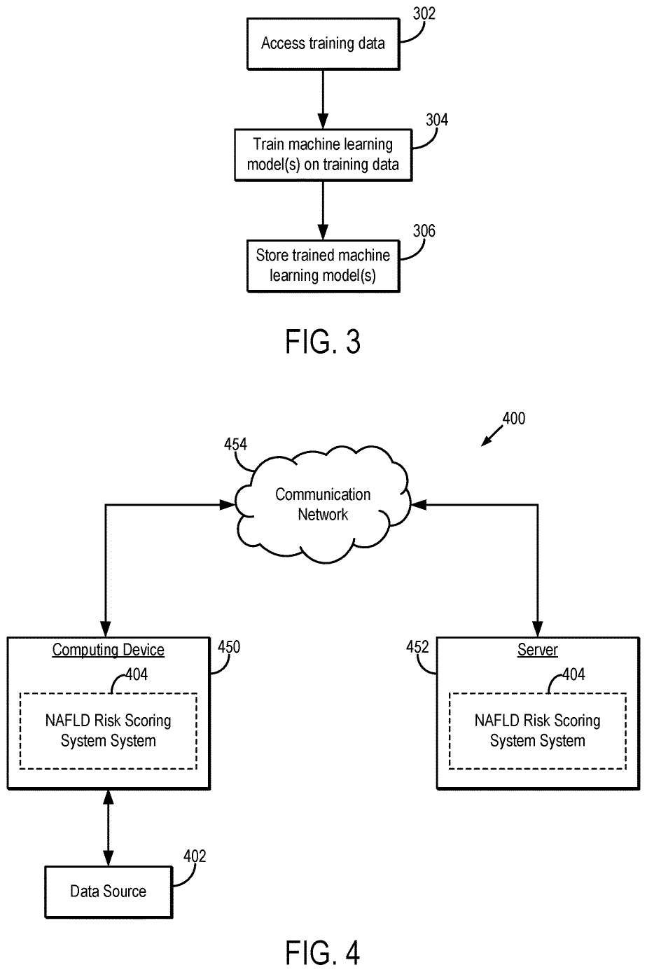

Referring now to FIG. 3, a flowchart is illustrated as setting forth the steps of an example method for training one or more machine learning models on training data, such that the one or more neural networks are trained to receive patient health data as input data in order to generate NAFLD risk score data as output data, where the NAFLD risk score data are indicative of a percent score, a probability, a numerical score, and/or a categorical indicator (e.g., “high” risk, “moderate” risk, “low” risk) for being at risk for NAFLD. Additionally or alternatively, the NAFLD risk score data can include a quantitative estimate of tissue and/or organ damage, such as how severe damage is (mild, moderate, advanced), a stage of scar tissue, the presence of liver cirrhosis, and so on.

In general, the machine learning model(s) can implement any number of different architectures. For example, as described above, the machine learning model(s) may include decision tree-based models (e.g., random forest, GBM) and/or neural networks. When a neural network is used, any number of different neural network architectures may be implemented. For instance, the neural network(s) could implement a convolutional neural network, a residual neural network, or the like.

The method includes accessing training data with a computer system, as indicated at step 302. Accessing the training data may include retrieving such data from a memory or other suitable data storage device or medium. In general, the training data can include patient health data acquired from a cohort or population of patients. In some embodiments, the training data may include patient health data sets that have been labeled (e.g., labeled as being associated with a clinical diagnosis of NAFLD, labeled as being associated with a particular severity of NAFLD, and so on). Thus, in some embodiments, the training data can include pairs of inputs (patient health data features) and outputs (clinical diagnoses, disease severity) such that a supervised learning technique can be used when training the machine learning models. Alternatively, unsupervised or other learning techniques may also be implemented.

As a non-limiting example, the training data can include an EHR dataset of 97,000 patients with NAFLD and 380,000 individuals without NAFLD, which can be used to train and validate machine learning models, such as one model to identify patients with NAFLD and another model to recognize NAFLD at risk of progression towards cirrhosis and liver-related events. For this latter model, the outcomes can be represented by development of cirrhosis, liver decompensation events (ascites, esophageal variceal bleeding, hepatic encephalopathy, jaundice), liver cancer, liver transplantation and death. Both machine learning models can be trained on routinely collected patient health data during the individuals; healthcare (demographics, anthropometrics, laboratory values, diagnoses, and medications, and others described above), which makes it generalizable to various different EHR systems. The machine learning model(s) can be trained to identify complex processes and patterns without a human's guidance and discover early comorbidity clusters that reflect a phenotype at risk to develop NAFLD later in life and to further stratify patients into subgroups with different disease trajectories (phenotypes). As a non-limiting example, the cohort can be split into training (70%), testing (20%) and validation (10%) groups.

The method can also include assembling training data from the cohort of patient health data using a computer system. This step may include assembling the patient health data into an appropriate data structure on which the machine learning model can be trained. Assembling the training data may include assembling patient health data and other relevant data. For instance, assembling the training data may include generating labeled data and including the labeled data in the training data. Labeled data may include patient health data or other relevant data that have been labeled as belonging to, or otherwise being associated with, one or more different classifications or categories. For instance, labeled data may include patient health data that have been labeled as being associated with a diagnosis of NAFLD, one or more severity stages, and so on.

One or more machine learning models are trained on the training data, as indicated at step 304. In general, the machine learning model can be trained by optimizing model parameters (e.g., weights, biases, or both) based on minimizing a loss function. As one non-limiting example, the loss function may be a mean squared error loss function.

Training a machine learning model may include initializing the model, such as by computing, estimating, or otherwise selecting initial model parameters (e.g., weights, biases, or both). In the example of training a neural network, during training, an artificial neural network receives the inputs for a training example and generates an output using the bias for each node, and the connections between each node and the corresponding weights. For instance, training data can be input to the initialized neural network, generating output as NAFLD risk score data. The artificial neural network then compares the generated output with the actual output of the training example in order to evaluate the quality of the NAFLD risk score data. For instance, the NAFLD risk score data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The machine learning model can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the machine learning model is configured to learn a general rule or model that maps the inputs to the outputs based on the provided example input-output pairs.

The one or more trained machine learning models are then stored for later use, as indicated at step 306. Storing the machine learning model(s) may include storing model parameters (e.g., weights, biases, or both), which have been computed or otherwise estimated by training the machine learning model(s) on the training data. Storing the trained machine learning model(s) may also include storing the particular model architecture to be implemented. For instance, data pertaining to the layers in the neural network architecture (e.g., number of layers, type of layers, ordering of layers, connections between layers, hvperparameters for layers) may be stored.

Referring now to FIG. 4, an example of a system 400 for NAFLD risk stratification in accordance with some embodiments of the systems and methods described in the present disclosure is shown. As shown in FIG. 4, a computing device 450 can receive one or more types of data (e.g., patient health data) from data source 402. In some embodiments, computing device 450 can execute at least a portion of a NAFLD risk scoring system 404 to generate NAFLD risk score data from patient health data received from the data source 402.

Additionally or alternatively, in some embodiments, the computing device 450 can communicate information about data received from the data source 402 to a server 452 over a communication network 454, which can execute at least a portion of the NAFLD risk scoring system 404. In such embodiments, the server 452 can return information to the computing device 450 (and/or any other suitable computing device) indicative of an output of the NAFLD risk scoring system 404.

In some embodiments, computing device 450 and/or server 452 can be any suitable computing device or combination of devices, such as a desktop computer, a laptop computer, a smartphone, a tablet computer, a wearable computer, a server computer, a virtual machine being executed by a physical computing device, and so on. The computing device 450 and/or server 452 can also reconstruct images from the data.

In some embodiments, data source 402 can be any suitable source of data, such as an EHR system or another computing device (e.g., a server storing patient health data), and so on. In some embodiments, data source 402 can be local to computing device 450. For example, data source 402 can be incorporated with computing device 450 (e.g., computing device 450 can be configured as part of a device for measuring, recording, estimating, acquiring, or otherwise collecting or storing data). As another example, data source 402 can be connected to computing device 450 by a cable, a direct wireless link, and so on. Additionally or alternatively, in some embodiments, data source 402 can be located locally and/or remotely from computing device 450, and can communicate data to computing device 450 (and/or server 452) via a communication network (e.g., communication network 454).

In some embodiments, communication network 454 can be any suitable communication network or combination of communication networks. For example, communication network 454 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced. WiMAX, etc.), other types of wireless network, a wired network, and so on. In some embodiments, communication network 454 can be a local area network, a wide area network, a public network (e.g., the Internet), a private or semi-private network (e.g., a corporate or university intranet), any other suitable type of network, or any suitable combination of networks. Communications links shown in FIG. 4 can each be any suitable communications link or combination of communications links, such as wired links, fiber optic links, Wi-Fi links. Bluetooth links, cellular links, and so on.

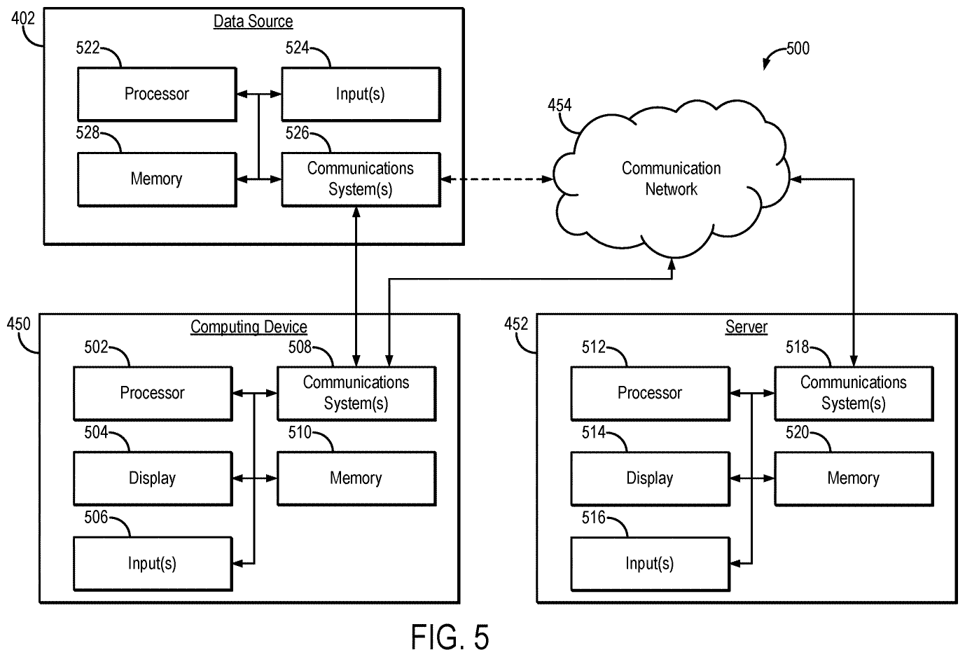

Referring now to FIG. 5, an example of hardware 500 that can be used to implement data source 402, computing device 450, and server 452 in accordance with some embodiments of the systems and methods described in the present disclosure is shown.

As shown in FIG. 5, in some embodiments, computing device 450 can include a processor 502, a display 504, one or more inputs 506, one or more communication systems 508, and/or memory 510. In some embodiments, processor 502 can be any suitable hardware processor or combination of processors, such as a central processing unit (“CPU”), a graphics processing unit (“GPU”), and so on. In some embodiments, display 504 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 506 can include any suitable input devices and/or sensors that can be used to receive user input, such as a keyboard, a mouse, a touchscreen, a microphone, and so on.

In some embodiments, communications systems 508 can include any suitable hardware, firmware, and/or software for communicating information over communication network 454 and/or any other suitable communication networks. For example, communications systems 508 can include one or more transceivers, one or more communication chips and/or chip sets, and so on. In a more particular example, communications systems 508 can include hardware, firmware, and/or software that can be used to establish a Wi-Fi connection, a Bluetooth connection, a cellular connection, an Ethernet connection, and so on.

In some embodiments, memory 510 can include any suitable storage device or devices that can be used to store instructions, values, data, or the like, that can be used, for example, by processor 502 to present content using display 504, to communicate with server 452 via communications system(s) 508, and so on. Memory 510 can include any suitable volatile memory, non-volatile memory, storage, or any suitable combination thereof. For example, memory 510 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 510 can have encoded thereon, or otherwise stored therein, a computer program for controlling operation of computing device 450. In such embodiments, processor 502 can execute at least a portion of the computer program to present content (e.g., images, user interfaces, graphics, tables), receive content from server 452, transmit information to server 452, and so on. For example, the processor 502 and the memory 510 can be configured to perform the methods described herein (e.g., the method of FIG. 1, the method of FIG. 3).

In some embodiments, server 452 can include a processor 512, a display 514, one or more inputs 516, one or more communications systems 518, and/or memory 520. In some embodiments, processor 512 can be any suitable hardware processor or combination of processors, such as a CPU, a GPU, and so on. In some embodiments, display 514 can include any suitable display devices, such as an LCD screen, LED display, OLED display, electrophoretic display, a computer monitor, a touchscreen, a television, and so on. In some embodiments, inputs 516 can include any suitable input devices and/or sensors that can be used to receive user input, such as a keyboard, a mouse, a touchscreen, a microphone, and so on.

In some embodiments, communications systems 518 can include any suitable hardware, firmware, and/or software for communicating information over communication network 454 and/or any other suitable communication networks. For example, communications systems 518 can include one or more transceivers, one or more communication chips and/or chip sets, and so on. In a more particular example, communications systems 518 can include hardware, firmware, and/or software that can be used to establish a Wi-Fi connection, a Bluetooth connection, a cellular connection, an Ethernet connection, and so on.

In some embodiments, memory 520 can include any suitable storage device or devices that can be used to store instructions, values, data, or the like, that can be used, for example, by processor 512 to present content using display 514, to communicate with one or more computing devices 450, and so on. Memory 520 can include any suitable volatile memory, non-volatile memory, storage, or any suitable combination thereof. For example, memory 520 can include RAM. ROM, EPROM. EEPROM, other types of volatile memory, other types of non-volatile memory, one or more types of semi-volatile memory, one or more flash drives, one or more hard disks, one or more solid state drives, one or more optical drives, and so on. In some embodiments, memory 520 can have encoded thereon a server program for controlling operation of server 452. In such embodiments, processor 512 can execute at least a portion of the server program to transmit information and/or content (e.g., data, images, a user interface) to one or more computing devices 450, receive information and/or content from one or more computing devices 450, receive instructions from one or more devices (e.g., a personal computer, a laptop computer, a tablet computer, a smartphone), and so on.

In some embodiments, the server 452 is configured to perform the methods described in the present disclosure. For example, the processor 512 and memory 520 can be configured to perform the methods described herein (e.g., the method of FIG. 1, the method of FIG. 3).

In some embodiments, data source 402 can include a processor 522, one or more input 524, one or more communications systems 526, and/or memory 528. In some embodiments, processor 522 can be any suitable hardware processor or combination of processors, such as a CPU, a GPU, and so on. In some embodiments, the one or more inputs 524 are generally configured to collect or otherwise receive patient health data, and can include an EHR system to which a user inputs recorded patient health data values. Additionally or alternatively, in some embodiments, the one or more inputs 524 can include any suitable hardware, firmware, and/or software for coupling to and/or controlling operations of an EHR system, or the like. In some embodiments, one or more portions of the inputs(s) 524 can be removable and/or replaceable.

Note that, although not shown, data source 402 can include any suitable inputs and/or outputs. For example, data source 402 can include input devices and/or sensors that can be used to receive user input, such as a keyboard, a mouse, a touchscreen, a microphone, a trackpad, a trackball, and so on. As another example, data source 402 can include any suitable display devices, such as an LCD screen, an LED display, an OLED display, an electrophoretic display, a computer monitor, a touchscreen, a television, etc., one or more speakers, and so on.

In some embodiments, communications systems 526 can include any suitable hardware, firmware, and/or software for communicating information to computing device 450 (and, in some embodiments, over communication network 454 and/or any other suitable communication networks). For example, communications systems 526 can include one or more transceivers, one or more communication chips and/or chip sets, and so on. In a more particular example, communications systems 526 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 528 can include any suitable storage device or devices that can be used to store instructions, values, data, or the like, that can be used, for example, by processor 522 to control the one or more data acquisition systems 524, and/or receive data from the one or more data acquisition systems 524: to generate images from data; present content (e.g., data, images, a user interface) using a display; communicate with one or more computing devices 450; and so on. Memory 528 can include any suitable volatile memory, non-volatile memory, storage, or any suitable combination thereof. For example, memory 528 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 528 can have encoded thereon, or otherwise stored therein, a program for controlling operation of data source 402. In such embodiments, processor 522 can execute at least a portion of the program to generate images, transmit information and/or content (e.g., data, images, a user interface) to one or more computing devices 450, receive information and/or content from one or more computing devices 450, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM. EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for risk stratifying a patient for non-alcoholic fatty liver disease using machine learning, comprising:
accessing patient health data for a patient with a computer system;
accessing a machine learning model with the computer system, wherein the machine learning model has been trained on training data in order to generate non-alcoholic fatty liver disease (NAFLD) risk scores based on features present in a patient's patient health data;
applying the patient health data to the machine learning model, generating an output as NAFLD risk score data that indicate a risk of the patient developing NAFLD based on features in their patient health data.

2. The method of claim 1, wherein the machine learning model comprises a decision tree-based machine learning model.

3. The method of claim 2, wherein the decision tree-based machine learning model is a gradient boosting machines (GBM) model.

4. The method of claim 1, wherein the machine learning model comprises an artificial neural network.

5. The method of claim 4, wherein the artificial neural network is a convolutional neural network.

6. The method of claim 1, further comprising selecting a subset of features from the patient health data and inputting only the subset of features to the machine learning model.

7. The method of claim 6, wherein the subset of features is determined by training another machine learning model on patient health data collected from a cohort of patients.

8. The method of claim 6, wherein the subset of features comprises patient demographics, anthropometrics, laboratory values, diagnoses, and medications.

9. The method of claim 6, wherein the subset of features includes patient age at diagnosis.

10. The method of claim 6, wherein the subset of features includes glucose levels measured when the patient was fasting.

11. The method of claim 6, wherein the subset of features includes laboratory values obtained from a blood test of the patient.

12. The method of claim 11, wherein the laboratory values comprise at least one of a blood urea nitrogen value, an anion gap value, an alanine transaminase value, an aspartate transferase value, a triglyceride value, a thyroid-stimulating hormone value, or an alkaline phosphatase value.

13. The method of claim 1, further comprising generating an order set based on analyzing the NAFLD risk score data with the computer system and storing the order set in an electronic health record (EHR) system.

14. The method of claim 13, wherein the order set comprises orders for additional testing for the patient based on an indicate level of risk for developing NAFLD determined by the NAFLD risk score data.

15. The method of claim 1, wherein the NAFLD risk score data comprise probability values for developing NAFLD.

16. The method of claim 1, wherein the NAFLD risk score data comprise category labels indicating low, moderate, or high risk for developing NAFLD.

17. The method of claim 1, wherein the NAFLD risk score data comprise quantitative estimates of tissue damage.

18. The method of claim 17, wherein the quantitative estimates of tissue damage include a severity score of tissue damage comprising a value of mild severity, moderate severity, or advanced severity.

19. The method of claim 17, wherein the quantitative estimates of tissue damage comprise scar tissue staging values.
